(19) United States
(12) Patent Application Publication
(10) Pub. No.: US 2024/0090772 A1
(43) Pub. Date: Mar. 21, 2024

(54) Intraoral Diagnostic Device and Method of Using Same

(71) Applicant: Colgate-Palmolive Company, New York, NY (US)

(72) Inventors: Hrebesh Molly SUBHASH, Somerset, NJ (US); Benny E. URBAN, JR., Somerset, NJ (US)

(73) Assignee: Colgate-Palmolive Company, New York, NY (US)

(21) Appl. No.: 18/262,062

(22) PCT Filed: Jan. 6, 2022
(86) PCT No.: PCT/US2022/011370

§ 371 Date: Jul. 19, 2023

Related U.S. Application Data
(60) Provisional application No. 63/139,360, filed on Jan. 20, 2021.

(51) Int. Cl. A61B 5/00

(52) U.S. Cl. CPC A61B 5/0088; A61B 5/0075; A61B 5/682; A61B 2560/0233; A61B 2562/0233; A61B 2562/046; A61B 2576/02

(57) ABSTRACT

An intraoral device (100) for determining oral health characteristics is provided. The device includes a light source (106) configured to emit light in a plurality of wavelengths within or about the oral cavity and a matrix array multispectral sensor (102) configured to detect a plurality of spectral channels of a spectral image. Each of the plurality of spectral channels may allow transmission of a corresponding wavelength. The device may include a processor (1100) configured to identify the detected plurality of spectral channels of the spectral image relating to the oral cavity. Based on the detected plurality of spectral channels, the processor may cause the light source to adjust the light emitted within or about the oral cavity to modify a signal to noise ratio and/or an image calibration of a subsequent spectral image. The matrix array multispectral sensor may capture the subsequent spectral image relating the oral cavity in the adjusted emitted light.

### CROSS-REFERENCE TO RELATED APPLICATIONS

The present application claims priority to U.S. Provisional Patent Application Ser. No. 63/139,360, filed Jan. 20, 2021, the entirety of which is incorporated herein by reference.

### BACKGROUND

Technology for promoting a user's health is known. In particular, connected health and consumer level diagnostic devices have been used among consumers to improve their long-term health care. For example, biometric devices may be used to ensure users are walking around and moving enough to prevent long-term muscular-skeletal problems and other health conditions.

Comprehensive consumer level biometric and diagnostic devices for oral hygiene, however, are not commonly known or available. Consumer level oral diagnostic devices have been introduced, but these devices are often lacking in functionality for improving oral health, such as for caries detection, plaque detection, functional blood mapping, artificial intelligence (AI) network diagnostics, longitudinal monitoring of hygiene, whiteness measurements, hydration measurements, tongue bacteria monitoring, and health product recommendations. Further, none of the current devices receive input from the user about their current oral health problems or oral health goals. Thus, a simple and easy to use platform that allows connected and automated longitudinal monitoring of oral health is desired.

### BRIEF SUMMARY

The present disclosure may be directed, in one aspect, to an intraoral device for determining oral health characteristics. The device includes a light source configured to emit light in a plurality of wavelengths within or about the oral cavity and a matrix array multispectral sensor configured to detect a plurality of spectral channels of a spectral image. Each of the plurality of spectral channels may allow transmission of a corresponding wavelength. The device may include a processor configured to identify the detected plurality of spectral channels of the spectral image relating to the oral cavity. Based on the detected plurality of spectral channels, the processor may cause the light source to adjust the light emitted within or about the oral cavity to modify a signal to noise ratio and/or an image calibration of a subsequent spectral image. The matrix array multispectral sensor may capture the subsequent spectral image relating the oral cavity in the adjusted emitted light.

In another aspect, a method for determining the oral health characteristics of an oral cavity is provided. The method includes emitting, via a light source, light in a plurality of wavelengths within or about the oral cavity and detecting, via a matrix array multispectral sensor, a plurality of spectral channels of a spectral image relating to the oral cavity receiving the emitted light. Each of the plurality of spectral channels may allow transmission of a corresponding wavelength. The method further includes identifying the detected plurality of spectral channels of the spectral image relating to the oral cavity, and, based on the detected plurality of spectral channels, causing the light source to adjust the light emitted within or about the oral cavity to modify at least one of a signal to noise ratio or an image calibration of a subsequent spectral image. The method further includes causing the matrix array multispectral sensor to capture a subsequent spectral image relating the oral cavity in the adjusted emitted light.

### BRIEF DESCRIPTION OF THE DRAWINGS

The present disclosure will become more fully understood from the detailed description and the accompanying drawings, wherein:

FIG. 1 is a front perspective view of an example intraoral insert device, as described herein.

FIGS. 2A-2C show top, bottom, and bottom-side views of the example intraoral insert device, as shown on FIG. 1.

FIG. 2D shows an exploded top view of the example intraoral insert device, as shown on FIGS. 2A-2C.

FIG. 2E shows an exploded bottom-side view of the example intraoral insert device, as shown on FIGS. 2A-2C.

FIGS. 3A-3D show views of another example intraoral insert device, as described herein.

FIGS. 4A-4D show example reflector configurations for the intraoral insert device, as described herein.

FIG. 5 shows example pulsing of a fluorescent excitation diode, as described herein.

FIGS. 6A, 6B show example major chromospheres which absorb light in the visible region, as described herein.

FIGS. 7A-7D show example gingival tissue with an overlaying layer of water, as described herein.

FIGS. 8A-8B show example measurements of volume fraction of blood/tissue de-oxygen saturation and melanin map in gingival tissue, as described herein.

FIG. 9 shows an example tooth color measurement based on reflectance mode measurement, as described herein.

FIG. 10 shows an image from example matrix array multispectral sensor showing caries, biofilm (plaque), and a normal tooth (baseline).

FIG. 11 describes an example process for using the intraoral insert device, as described herein.

### DETAILED DESCRIPTION

The following description of the preferred embodiment(s) is merely exemplary in nature and is in no way intended to limit the invention or inventions. The description of illustrative embodiments is intended to be read in connection with the accompanying drawings, which are to be considered part of the entire written description. In the description of the exemplary embodiments disclosed herein, any reference to direction or orientation is merely intended for convenience of description and is not intended in any way to limit the scope of the present inventions. Relative terms such as “lower,” “upper,” “horizontal,” “vertical,” “above,” “below,” “up,” “down,” “left,” “right,” “top,” “bottom,” “front” and “rear” as well as derivatives thereof (e.g., “horizontally,” “downwardly,” “upwardly,” etc.) should be construed to refer to the orientation as then described or as shown in the drawing under discussion. These relative terms are for convenience of description only and do not require a particular orientation unless explicitly indicated as such.

Terms such as “attached,” “affixed,” “connected,” “coupled,” “interconnected,” “secured” and other similar terms refer to a relationship wherein structures are secured or attached to one another either directly or indirectly through intervening structures, as well as both movable or rigid attachments or relationships, unless expressly described otherwise. The discussion herein describes and illustrates some possible non-limiting combinations of features that may exist alone or in other combinations of features. Furthermore, as used herein, the term “or” is to be interpreted as a logical operator that results in true whenever one or more of its operands are true. Furthermore, as used herein, the phrase “based on” is to be interpreted as meaning “based at least in part on,” and therefore is not limited to an interpretation of “based entirely on.”

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Features of the present inventions may be implemented in software, hardware, firmware, or combinations thereof. The computer programs described herein are not limited to any particular embodiment, and may be implemented in an operating system, application program, foreground or background processes, driver, or any combination thereof. The computer programs may be executed on a single computer or server processor or multiple computer or server processors.

Processors described herein may be any central processing unit (CPU), microprocessor, micro-controller, computational, or programmable device or circuit configured for executing computer program instructions (e.g., code). Various processors may be embodied in computer and/or server hardware of any suitable type (e.g., desktop, laptop, notebook, tablets, cellular phones, etc.) and may include all the usual ancillary components necessary to form a functional data processing device including without limitation a bus, software and data storage such as volatile and non-volatile memory, input/output devices, graphical user interfaces (GUIs), removable data storage, and wired and/or wireless communication interface devices including Wi-Fi, Bluetooth, LAN, etc.

Computer-executable instructions or programs (e.g., software or code) and data described herein may be programmed into and tangibly embodied in a non-transitory computer-readable medium that is accessible to and retrievable by a respective processor as described herein which configures and directs the processor to perform the desired functions and processes by executing the instructions encoded in the medium. A device embodying a programmable processor configured to such non-transitory computer-executable instructions or programs may be referred to as a “programmable device”, or “device”, and multiple programmable devices in mutual communication may be referred to as a “programmable system.” It should be noted that non-transitory “computer-readable medium” as described herein may include, without limitation, any suitable volatile or non-volatile memory including random access memory (RAM) and various types thereof, read-only memory (ROM) and various types thereof, USB flash memory, and magnetic or optical data storage devices (e.g., internal/external hard disks, floppy discs, magnetic tape CD-ROM, DVD-ROM, optical disk, ZIP™ drive, Blu-ray disk, and others), which may be written to and/or read by a processor operably connected to the medium.

In certain examples, the present inventions may be embodied in the form of computer-implemented processes and apparatuses such as processor-based data processing and communication systems or computer systems for practicing those processes. The present inventions may also be embodied in the form of software or computer program code embodied in a non-transitory computer-readable storage medium, which when loaded into and executed by the data processing and communications systems or computer systems, the computer program code segments configure the processor to create specific logic circuits configured for implementing the processes.

Health and/or diagnostic devices (e.g., connected health and/or diagnostic devices) may be used by consumers concerned with their short and/or long-term health care. One or more oral diagnostic devices (e.g., consumer level oral diagnostic devices) may be described herein. These devices may provide caries detection, plaque detection, functional blood mapping, artificial intelligence (AI) network diagnostics, longitudinal monitoring of hygiene, whiteness measurements, hydration measurements, tongue bacteria monitoring, and health/or product recommendations for improving oral health. Such devices may receive input from the user about the current oral health problems or oral health goals of the user.

Intraoral cameras (e.g., cost-effective intraoral cameras) may be coupled to oral diagnostic devices and/or may connect (e.g., wirelessly connect) to smart devices. Smart devices may include mobile phones, tablets, laptops, and the like. Intraoral cameras may allow users to capture (e.g., efficiently capture) RGB color images and/or video of the oral cavity of the user. The images and/or video may be sent to one or more persons (such as an oral care professional) to diagnose oral tissue health and hygiene. Acquiring high quality images of the oral cavity (e.g., the whole oral cavity) may be complicated, for example, based on lighting conditions and/or consumer skill levels. Further, non-professionals (such as consumers of oral care devices) may not know the best camera focus, angle, and/or lighting conditions.

One or more RGB cameras may not have the same ‘red,’ ‘green,’ and ‘blue’ spectral detection abilities or illumination spectra, creating non-true color values and color variations between intraoral imaging devices. Because color is a significant indicator of tissue health, non-true color values can lead to misdiagnosis of oral care issues. Further, from the diagnostic side, oral care professionals who receive intraoral images and/or videos on a connected platform are tasked with sorting through large amounts of image data and making visual diagnosis. This may be time consuming for busy professionals that need to see numerous patients and accurately make decisions that will affect their health.

A system, device, and/or method may incorporate one or more matrix array multispectral sensors (e.g., matrix array multispectral detector), one or more light sources, and/or one or more reflective elements. Such system, device, and/or method may overcome the disadvantages described above. The system, device, and/or method may include a device that may be inserted into a user's oral cavity, such as a wearable oral device (e.g., tray). The device may include one or more matrix array multispectral sensors, one or more light sources, and/or one or more reflective elements. The matrix array multispectral sensor may capture mouth images, such as whole mouth spectral images. The light source may illuminate the oral cavity.

Spectral imaging may be imaging that uses multiple bands across the electromagnetic spectrum. For example, an ordinary camera may capture light across three wavelength bands in the visible spectrum (such as red, green, and blue (RGB)). Spectral imaging, on the other hand, may encompass a wide variety of techniques that go beyond RGB. For example, spectral imaging may use the infrared, the visible spectrum, the ultraviolet, x-rays, or some combination of infrared, the visible spectrum, the ultraviolet, x-rays. Spectral imaging may include the acquisition of image data in visible and non-visible bands simultaneously, illumination from outside the visible range, and/or the use of optical filters to capture a specific spectral range. Spectral imaging may capture hundreds of wavelength bands for each pixel in an image. The spectral images (e.g., produced via spectral imaging) may be analyzed using AI to observe hygiene, determine tissue health, and/or measure (e.g., longitudinally measure) quantitative changes in oral health.

The device (e.g., wearable device) may be capable of real-time mapping of plaque, potential caries/cavity spots, bleeding tissue detection, teeth whiteness measurements, blood pressure, heart rate, blood flow, ulcers, cracked teeth, over brushing determination, gingivitis mapping, biofilm, inflammation measurements, receding gums, periodontitis, tonsillitis, bad breath due to tongue bacteria diagnostics, soft-tissue melanin mapping, tissue hydration measurements/dry mouth, blood/tissue oxy-deoxygenation mapping for more comprehensive tissue health diagnostics, and the like. In examples, the device may include one or more other sensors and/or optical radiation sources (such as gas sensors for breath, motion sensors for motion artifact correction, temperature sensor for fever detection, ultrasonic transducers for tissue/blood monitoring, thermal camera for fever and inflammation mapping, etc.). The one or more other sensors and/or optical radiation sources may provide comprehensive health monitoring (e.g., comprehensive oral health monitoring) using one or more of reflection, absorption, transmission and fluorescence radiation. For example, quantitative hydration information can be discerned using 1450 nm and 1050 nm infrared reflection measurements and determining the ratio of absorbed light at each wavelength. An example for transmission mode may include determining early caries using SWIR transillumination.

Diagnostic results may be displayed (e.g., immediately displayed) to a user. Other information may be displayed to the user, such as potential health implications, dental visit suggestions, and/or hygiene product recommendations. Diagnostics may be saved to a smart device and/or sent (e.g., sent directly) to oral care professionals allowing for longitudinal monitoring of tissue health and/or hygiene progress. As the device may be inserted and/or worn within the oral cavity, the location of the tissues may be consistent during longitudinal imaging. Changes in lighting condition may be accounted for, for example, via a built in color calibrator. In examples where a smart device is used with an application (such as a smart-phone App), an optional virtual assistant may be activated to provide advice on improving oral health. The advice on improving oral health may be based on AI diagnostics. For example, a virtual assistant may suggest brushing techniques, an oral mouth rinse to treat ulcers in the mouth, usage instructions of oral care products, and the like. Improved image acquisition and AI diagnostics may simplify and/or improve the connected health platform for the user, may allow for longitudinal monitoring of health, may make health recommendations, may improve oral hygiene, etc.

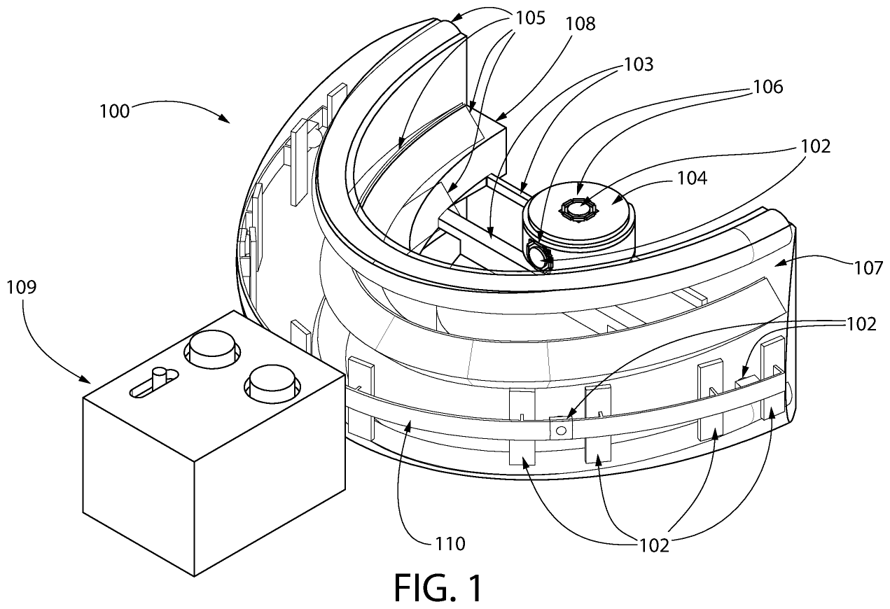

Referring now to the figures, FIG. 1 shows a schematic of an example diagnostic device 100 with multiple matrix array multispectral sensors. As shown on FIG. 1, the device 100 may be inserted into an oral cavity (e.g., an intraoral insert). The device 100 may house one or more matrix array multispectral sensors 102, such as a camera (e.g., an RGB or RGB-D camera). The matrix array multispectral sensor 102 may be suspended on a mount 103 that may be located inside and/or outside the device 100. The matrix array multispectral sensor 102 may have coupled optics (e.g., fisheye lenses) for ultrawide, close-up large field-of-view (FOV) imaging. To increase imaging FOV, the matrix array multispectral sensor 102 may be positioned on rotational stage 104 (e.g., top, bottom, and/or side(s) of rotational stage 104). The matrix array sensor may have distance measuring capabilities, such as ToF or active stereo vision as an example.

One or more matrix array multispectral sensors 102 may be incorporated to cover one or more portions of the oral cavity and/or the full oral cavity. In the example of the device 100 shown on FIG. 1, five external large FOV matrix array multispectral sensors 102 may be mounted on the outside wall of the device 100 to capture images of the front of the oral cavity. As shown on FIG. 1, one or more (e.g., two) matrix array multispectral sensors 102 may be mounted on the top and/or bottom of the movable mount to allow for detection of tissue properties of the roof of the mouth and/or the tongue. To allow for imaging of front, back and under teeth and soft tissues on a minimum number of matrix array devices, reflector elements 105 may be incorporated into the device insert. In examples, device 100 may include one or more accelerometers, gyroscopes, and the like. The accelerometers and/or gyroscopes may be used to determine motion, correct for motion, etc. For example, the accelerometers and/or gyroscopes may be used to determine motion and/or correct for motion in diagnostic images.

Reflective elements (e.g., located under a bite bar) may be positioned in such a way to reflect one or more portions of the teeth (such as the bottom of the teeth) into the imaging FOV of the high resolution moveable matrix array spectral sensor. Outer reflective elements may be positioned to give the external matrix array spectral sensors a clear view of portions in the oral cavity, such as the gingival tissues. The reflectors in combination with the matrix array multispectral sensors 102 may give a full view of the hard tissues, gingival tissues, large portions of the tongue, top of the mouth, tonsils, and the like. Some reflective elements can be reflective for some wavelengths and transmissive for other wavelengths to allow for transmission measurements of specific wavelengths. For example, a reflective element may have a dichroic coating that reflects visible light but allows SWIR light to transmit un-impeded. The un-impeded light being directly incident on tissue and being detected in a transmission mode.

The device 100 may contain light sources 106, which may be light emitting diodes (LEDs) (e.g., standard LEDs, organic LEDs, etc.), superluminescent diodes (SLEDs), lasers, arc lamps, a combination of the aforementioned radiation sources, other compact light sources for illuminating the oral cavity, etc. The light sources 106 may be located on one or more portions of device 100, such as on rotational stage 104. For example, the light sources 106 may be located on a top, bottom, and/or side(s) of rotational stage 104. The matrix array multispectral sensor 102 and/or the light sources 106 may have polarizers to cross-polarize the light (e.g., light provided by the light sources), which may remove specular reflection incident on the matrix array multispectral sensor. The light sources 106 may be broadband and/or near single wavelength. The illumination radiation may range from UV to SWIR. The light sources 106 may incorporate diffuser elements and/or other optics to shape the illumination light for even or patterned illumination. The body of the device 100 may be composed of a biocompatible transparent material 107 that may allow light to be transmitted.

A transparent bite bar, tooth rest 108, and/or tongue retractor may be incorporated into the device 100 for the user to place their teeth and tongue, which may promote consistent imaging. Small color squares (e.g., of known spectral absorption) may be imbedded in the transparent material and/or incorporated into the FOV of the matrix array spectral sensor for color calibration. The light sources 106 and/or matrix array sensor may be connected (e.g., electronically connected) to a control module 109 through an electronic support band 110.

The control module may contain one or more system-on-a-chips (SoCs). The SoC may be connected to a power supply (e.g., battery), such as an independent, rechargeable power supply. The power supply may be recharged through a cable and/or an inductance charging unit. In some examples the SoC may be wirelessly connected to a smart device and/or an external server (e.g., the Internet/Cloud). In some examples the SoC may be self-contained with the AI program implemented directly from the SoC. In other examples, pre-processing (such as color calibration) may be performed on the SoC and/or the pre-processed data may be sent to an external processor with an AI diagnostic algorithm. In the case of onboard AI, the SoC may be connected (e.g., wirelessly connected) to a smart user device or an external server (e.g., the Internet/Cloud) for updating the AI algorithm, updating the virtual assistant product and recommendation data base, logging longitudinal data, and/or sending data to a third party.

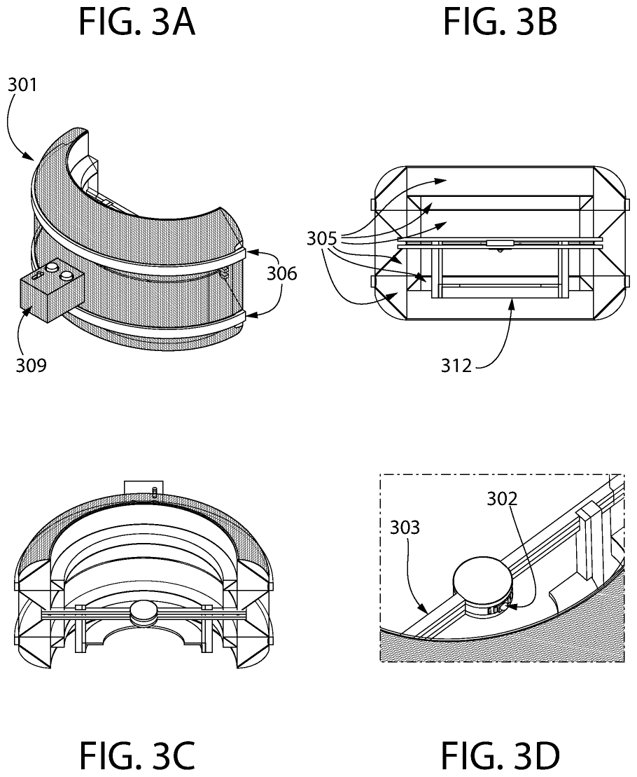

FIG. 3A shows another example of the device, such as device 301. In such example, the reflector components may be placed in locations which may allow for minimizing the number of matrix array imaging devices. FIG. 3A shows an example device 301 having a control module 309 in which the outer surface is coated in a material, such as a black biocompatible material (depicted in hatched lines). The material may be configured to reduce stray light (e.g., emitted from light source 306) from entering the sensor (e.g., matrix array sensor). FIG. 3B shows a back view of the device 301 in which multiple reflector elements 305 may be seen. The reflector elements 305 may be positioned in such a way as to reflect the front and/or bottom sides of the tissue (e.g., oral hard and soft tissues) into the matrix array sensor. In an example, the reflecting elements 305 may have flat surfaces, although in other examples the reflecting elements 305 may be concave or convex to increase FOV or resolution. FIG. 3C shows a top-back view of the device 301 in which the reflector elements 305 and matrix array imaging element may be seen. The reflector elements 305 and matrix array imaging element may be positioned on a turnable mount 303. The turnable mount 303 may have a rest (e.g., a tongue rest 312) fixed to the bottom of the mount 303. FIG. 3D shows a zoomed image of the matrix array sensor 302 and mount 303.

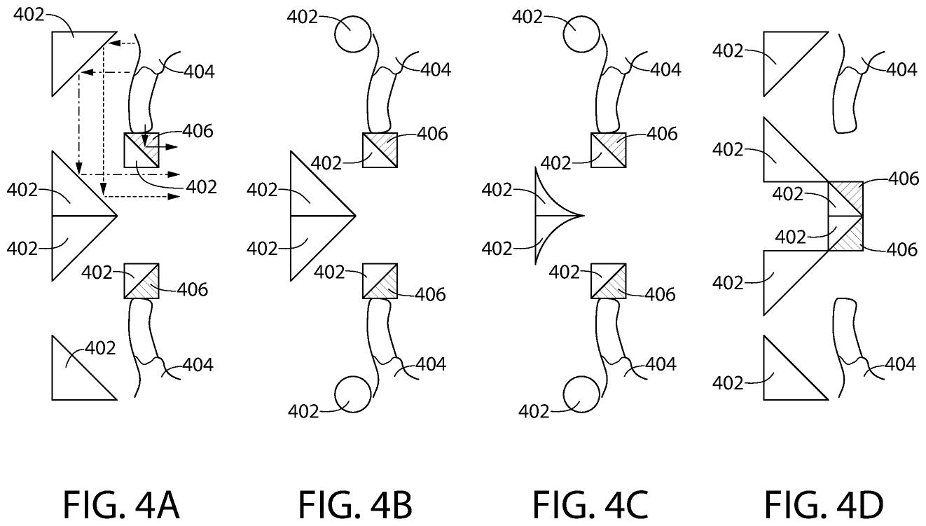

Example reflector configurations of the device (such as device 100) are shown in FIGS. 4A-4D. Examples of the device may have different illumination and/or reflector setups. The reflectors 402 may be used to reflect illumination from one or more objects, such as one or more tissues 404 (e.g., teeth). The tissue 404 may rest on a rest 406 (e.g., tooth rest) that may include transparent biocompatible material. In some examples, the reflectors 402 may be flat. Flat reflectors may prevent elongation or shrinkage of the imaged object (e.g., tissue 404) in the matrix array multispectral sensor 102. In some examples, the reflectors 402 may be concave or convex, for example, to adjust magnification or curvature issues. In some examples, the reflector 402 may allow for a single matrix array multispectral sensor to capture the entirety of the oral cavity.

Cameras may be employed in intraoral imaging devices (e.g., consumer level intraoral imaging devices) to capture images of hard to see tissue and/or to make diagnostic health evaluations. Diagnosing health from the devices may be complicated by the variations in light conditions, shadows, dirty optics, small FOV, different focusing depths, obstructed views, etc. These drawbacks may prevent high throughput determination of plaque, biometric diagnostics, and/or accurate position determination (e.g., what section of the mouth the image has captured) by quantitative algorithms and generally need medical experts for qualitative classification. Large image data sets may be cumbersome and/or time consuming for clinicians to diagnose in cases where patients need to send images to professionals for diagnostics. Different cameras may have different light sensitivities and/or spectral transmissions, which may make true color evaluations difficult or impossible. Real-time stitching algorithms may include one or more of the aforementioned problems, but may be currently available on a consumer level. Oral insert imaging devices may solve some complications (e.g., changes in focus and/or consistent illumination conditions), but these devices may primarily target hard tissue detection of plaque and may not target overall oral health.

Matrix array multispectral sensors 102, such as RGB, RGB-D, RGB-IR, multispectral, and hyperspectral cameras, may allow for a comprehensive evaluation of tissue health. Using a matrix array spectral sensor and/or calibrated light source, quantitative information relating to molecular fluorescence, light absorption, and/or light scattering may be derived. For example, spectrometers may be used to measure plaque on hard-tissue, bacteria on the tongue, teeth whiteness, and other biometric tissue health indicators (e.g., caries, bleeding, oxygenation, vascularization, dryness, oral cancer). With calibration, quantitative measurements of light fluorescence, tissue scattering, and/or tissue absorption may be achieved.

To overcome the drawbacks of light condition variation, image calibration using standard targets imbedded in the FOV of the matrix array sensor may be employed. The calibration may account for differences in camera sensitivities and/or spectral transmissions. To account for shadows, light element arrays may be employed to ensure full cavity illumination. Further, uneven illumination may account for by normalizing the illumination over the FOV of one or more (e.g., each) matrix array. To account for dirty optics, one or more (e.g., the majority) of the optical components may be embedded in a transparent, biocompatible polymer that may be rinsed with water or another cleaning material. To increase the FOV and/or capture normally obstructed views, large angle lenses, multiple matrix array multispectral sensors, and/or a moveable matrix array multispectral sensor may be used.

In the case of hygiene mapping, the captured diagnosed images may be stitched together to form a complete view (or 3D map) of the oral cavity (e.g., the entire oral cavity). The diagnostics may be displayed on a device (such as smartphone or tablet), with timing and/or orientation information from the set positions of the matrix array multispectral sensors being used to achieve position accuracy.

The device may incorporate squares (e.g., miniature color calibration squares) in the FOV of one or more (e.g., each) matrix array multispectral sensor. When the device is used (e.g., each time the device is used), the color checker may be used to calibrate the camera with a correction matrix to generate a true-color image and/or achieve sufficient spectral information for image diagnostics. The color checker may be used to determine the illumination intensity, for example, to ensure use of the full dynamic range of the matrix array multispectral sensor and improve signal-to-noise (SNR) for more accurate diagnostics. The color checker may be used for radiation(s) ranging from UV to SWIR wavelengths. The color checker may be embedded in the device and may occupy the location (e.g., same location) in the FOV of one or more (e.g., each) sensor. The location may be used in the calibration algorithm. The calibration algorithm may create a color correction matrix, which may be applied to one or more (e.g., each) spectral channel of the matrix array multispectral sensor. The result may allow for quantitative spectral absorption, reflection, and/or transmission measurements. Image color calibration may be handled by a preprocessing algorithm stored on the SoC, although in other examples image color calibration may be performed via an external server (e.g., using an external processor).

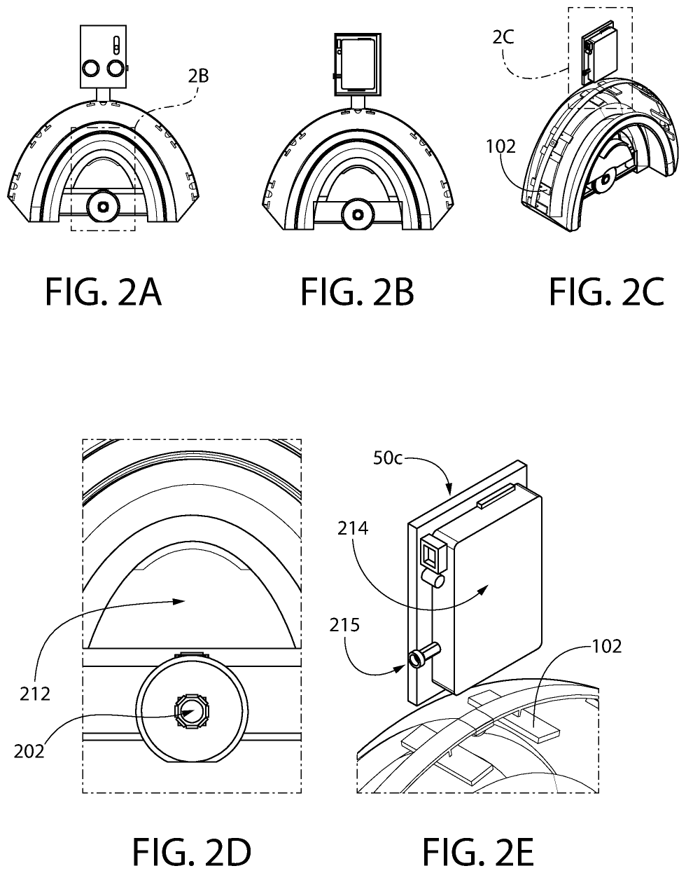

As shown in FIGS. 2A and 2C, the diagnostic device may be equipped with one or more onboard SoCs, such as SOCs 213. The SoC may be a microprocessor unit (MPU), a microcontroller unit (MCU), or a combination thereof. The MPU(s) and/or CPU(s) may be embedded in the control module of the device 109 (FIG. 1). The MPU(s) and/or CPU(s) may collect data from the matrix array sensors. The data may be used (e.g., used initially) to adjust the lighting conditions of the light elements, for example, to improve signal to noise ratio (SNR) and/or image calibrations before actual diagnostic images are captured. The matrix array spectral sensors may be embedded in the transparent polymer composing the body of the device. To homogenize the illumination light, a diffuser may be placed in front of the light sources.

LEDs may be used to illuminate the sample for spectral detection, although in examples other compact lighting elements may be incorporated instead or in addition. The MPU(s) and/or CPU(s) may have onboard wireless communication, such as Bluetooth or Wi-Fi, for streaming and/or receiving data. In some examples, data from the sensor may be directly streamed to an external device (such as an external server) for processing. In some examples the MPU(s) and/or CPU(s) may have memory for storing large amounts of data, which may be retrieved and/or streamed after device use. In another example, the onboard MPU(s) and/or CPU(s) may acquire and process data from the embedded sensor using an onboard AI. The onboard memory may contain accessible files for the machine learning algorithm training and/or for Monte Carlo data simulations for fitting scattering parameters to different melanin concentrations.

FIGS. 2A-2E show different views of the example device 100 shown on FIG. 1. FIG. 2D shows an exploded region of the top image. A transparent tongue retractor 212 may be provided and may include false colors for visibility. FIG. 2E shows an exploded region of the bottom-side of the device 100. As shown on FIG. 2E, the cover of the control module may be removed from the image to expose an SoC 213, power supply 214, and/or charging jack 215. The control module may house the power supply 214 for powering the matrix array multispectral sensors 102, lighting elements, MPU(s), and/or CPU(s). The power supply 214 may be attached to a charge controller. The charge controller may be attached (e.g., attached directly) to a removable power cable or an inductance charging coil. The output end of the battery may be attached to an onboard or power controller shim, for example, for controlling power into the connected architecture.

Consumer level intraoral imaging devices may incorporate one or more RGB cameras for imaging tissues. The cameras may provide qualitative information about tissue health of an oral cavity. For example, lesions of unusual color in tissues may be captured and/or visually diagnosed as likely unhealthy. Quantitative information, which may confirm tissue health, may be determined using sophisticated algorithms. The algorithms may rely on illumination (e.g., consistent illumination) conditions, near-flat surfaces, known spectra of the light source, known transmissions of the camera filters, consistent gain and integration settings, and non-adaptive color corrections. The requirements may complicate quantitative diagnostics.

The known spectral channels may be incorporated into the platform in order to obtain functional information from the oral hard and soft tissue, such as dental plaque, enamel health, tooth whiteness, caries and soft tissue parameters such as gingival color, bleeding, angiogenesis, carcinogenesis, blood and tissue oximetry information, and the like. Matrix array spectral sensors (e.g., with calibration targets) may be used for accurate determination of spectra and/or camera calibration despite the illumination conditions. Accurate determination of spectra and/or camera calibration may allow the device 100 to account for ambient light or other potential changes in lighting conditions that may alter lighting and detection properties throughout the life of the device 100. The matrix array multispectral sensor(s) may require one or more (e.g., at least three) spectral channels or one or more (e.g., three) illumination sources (e.g., distinct illumination sources) for a monochrome device to determine functional information.

The matrix array multispectral sensor may be a CCD, CMOS, InGaN, or Si based array sensor (e.g., detector). For faster imaging, the matrix array may incorporate (e.g., integrate) spectral channels that may allow (e.g., may each allow) the transmission of a corresponding wavelength (e.g., light of specific wavelengths to pass through). The spectral channels may allow the transmission of a corresponding wavelength via a filter (e.g., optical filter, bandpass filter), prism, grating, light guide, and the like. In an example, the spectral channels (e.g., each of the spectral channels) may separate corresponding wavelengths of light. The spectral channels may allow light of a specific wavelength and/or specific bandwidth to pass (e.g., pass through a bandpass filter). The spectral channels (e.g., each of the spectral channels) may detect light of a corresponding wavelength.

One or more (e.g., each) spectral channels may have a bandwidth between 1 to 250 nm. The spectral channels may be customized for the biomolecules of interest and/or adjusted to cover the entire emission spectra of the LEDs. In the example of monochrome matrix array multispectral sensors, the color of the light source may be changed and/or detected. In monochromatic matrix array multispectral sensors, there may be a single spectral channel that may range from 250 to 2300 nm. The quantum efficiency of the device may be known for one or more (e.g., each) wavelength and may be accounted for in the functional analysis. The acquired images may be diagnosed by an algorithm (such as an AI or machine learning algorithm) for extracting functional, aesthetic, tissue health, and hygiene oral information (such as plaque score, whiteness score, enamel health, gingival color information, tissue spectroscopy, pulse, blood pressure, temperature, bleeding, lesions, gingivitis, dry mouth, and other oral conditions) which may affect numerous people in the world.

The illumination system may have one or more (e.g., multiple) embodiments. In the examples shown on FIGS. 1 and 2A-E, the illumination system may include one or more (e.g., multiple) LEDs. The LEDs may provide multiple wavelengths, which may be provided individually during multiplexing the light sources or simultaneously by applying a continuous voltage and current to the light source. The LEDs may be multiplexed by illuminating them one at a time and acquiring intraoral images and/or the LEDs can be simultaneously illuminated, giving a near uniform illumination in the oral cavity. LEDs may be capable of emission at 405 nm for fluorescence measurement. Broadband LEDs can range from 390 to 2300 nm and may be incorporated for reflectance measurements.

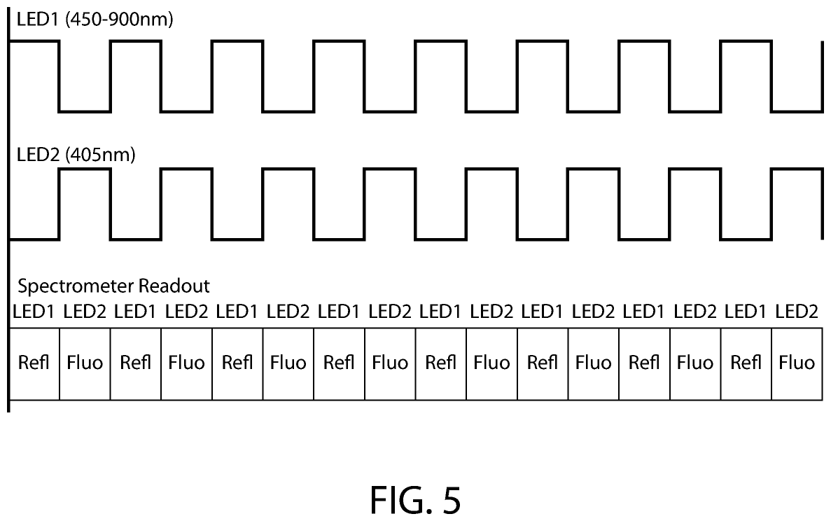

Fluorescence and/or reflectance data may be acquired by multiplexing the sensor at a high readout speed (e.g., greater than 10 Hz). Before calculating tissue properties, a reflectance standard consistently embedded in the same location in the device body may be measured to calibrate the device. The fluorescent excitation diode (e.g., UV/blue and broadband white light LED) may be pulsed, as shown in FIG. 5. Although a square wave is shown in FIG. 5, the actual pulsed wave may be any shape for driving the LEDs in the time frame desired. In addition, high intensity pulse with low average powers may be used to increase the signal-to-noise ratio and/or maintain low average powers that may not damage tissues. One or more (e.g., both) driving waves may be phase shifted, such that the illumination of the excitation and/or white light LED do not overlap. The time of the excitation and/or white light LEDs may be adjusted to optimize for signal as needed. Data may be acquired by the matrix array multispectral sensor at one or more (e.g., each) of the ‘ON’ LED states. The fluorescence and/or white light spectra may be separated using their respective time stamps.

A range of longitudinal and/or quantitative reflectance measurements may be performed with the visible-infrared bands for hard and/or soft tissue conditions, which may include oxy-deoxy and oxygen saturation of gum tissue, gum tissue hydration, blood pressure, heart rate, blood flow, inflammation measurements, melanin concentration, gum and tooth color change, early detection of gum bleeding, and the like.

Reflectance spectroscopy is non-invasive and may be used for soft and/or hard tissue characterization and/or early detection of various skin conditions. Reflectance spectroscopy may adopt diffusion, Monte Carlo, and/or other tissue model approximations to study the light propagation in the biological tissues. Because the measurement of reflectance spectroscopy may depend upon the various tissue parameters, a computational model may be developed to enumerate the physiologically correlative components of the tissues for detection and differentiation of biological samples. Modeling the diffuse reflectance may be used for interpreting measurements and/or extracting information contained in them. Models for the diffuse reflectance may be accurate over a broad range of spatial, temporal, and/or frequency scales to investigate the scattering medium. Moreover, the models may be intuitive and easy to implement.

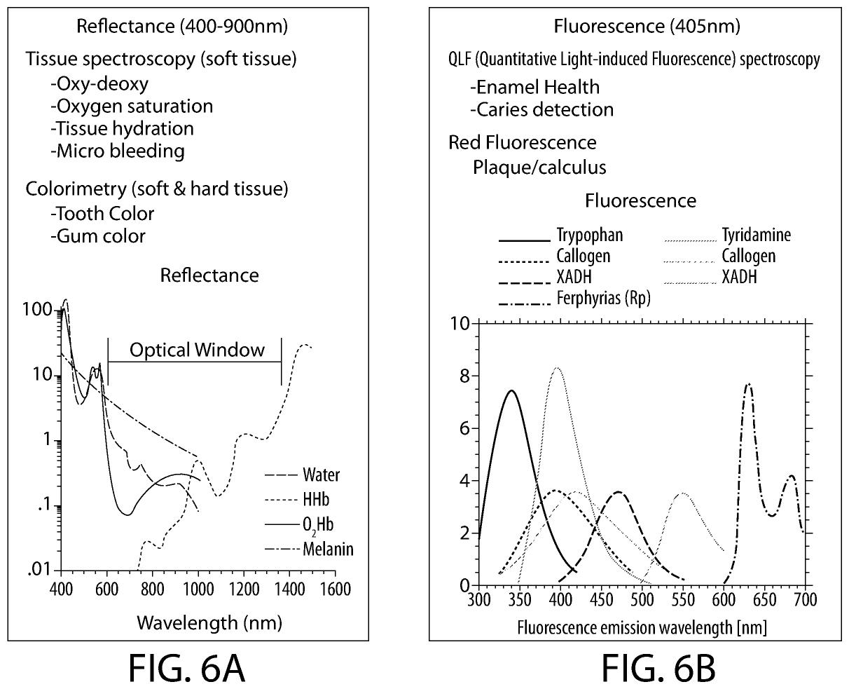

A diffusion approximation and/or Monte Carlo simulation may be used to solve and/or provide optical properties of tissues for diffuse reflectance applications. Diffusion approximation and/or Monte Carlo simulations may require stringent boundary conditions, such as the source-detector (e.g., sensor) separation being greater than the transport mean free path distance and/or the reduced scattering coefficient μs′ being greater than the absorption coefficient μa. The reduced scattering coefficient μs′ may be defined as the mean distance travelled by the photon before it gets scattered (absorbed). μs′ may be dependent on two variables: μs and g. The correlation may be as follows: μs′=μs (1−g). μs may be the cross-sectional area per unit volume of medium and g may be the amount of forward direction retained by the photon after a single scattering event. The major chromospheres which absorb light in the visible region (380-780 nm) may be oxygenated and/or deoxygenated hemoglobin (HbO and Hb respectively), as shown in FIGS. 6A, 6B.

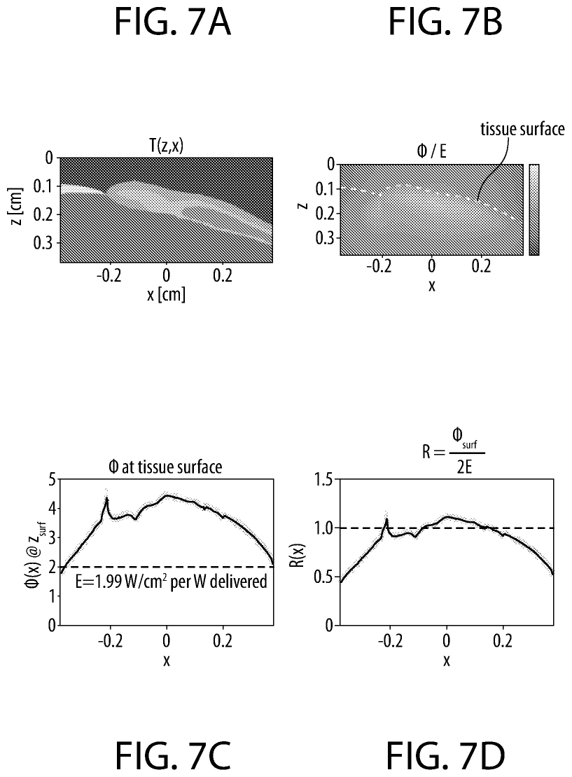

An optical tomography based structural morphology may be used for modeling the gingival tissue. FIGS. 7A-7D show an example gingival tissue with an overlaying layer of water. For this example, an 8 mm diameter beam may be used to yield a non-uniform reflectance. The beam may be delivered to a homogeneous tissue λ=810 nm; μa=0:5 cm⁻¹; μ′s=12.3 cm⁻¹, g=0.90. The reflectance may have edge losses when the irradiance is only as wide as the field of view.

The extinction coefficients for HbO and Hb may be obtained from known standards. For example, to calculate the Hb and HbO concentration from the values of μa the following equations may be used:

HbO2=((μa750*εHb830)−(μa830*εHb750))/(εHbO2 750*εHb830−εHbO2 830*εHb750)*1000

Hb=−((μa750*εHbO2 830)−(μa830*εHbO2 750))/(εHbO2 750*εHb830−εHbO2 830*εHb750)*1000

After obtaining the values, the range of μa for the respective wavelengths may be found.

The equation used to calculate the range of μa may be:

μa(λ)=[HbO2]*εHbO2(λ)+[Hb]*εHb(λ)+εH2O*% H2O

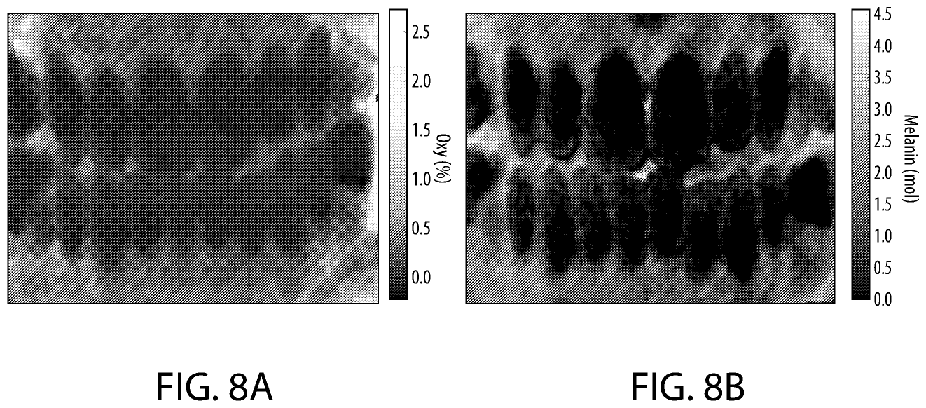

Using the above set of equations, the range (e.g., entire range) of the respective μa may be calculated for the different blood concentrations. FIGS. 8A-8B show an example measurement of volume fraction of blood/tissue de-oxygen saturation and/or melanin map in gingival tissue measured using a matrix array multispectral sensor system in reflectance mode.

In an example of the device (such as device 100), the matrix array spectral sensor may be used to acquire reflectance spectra from the tissue. In such examples, a broad band LED may be used to illuminate the tissue covering the spectral regions of interest (UV-SWIR). In another example, the matrix array multispectral sensor may be a monochrome detector with broad optical sensitivity in the spectral regions of interest and multiple light sources of known spectra can be illuminate the tissue at specific times. In such examples, the matrix array multispectral sensor may acquire images for one or more (e.g., each) light source. Because the spectra of the light source may be known, the reflection intensity may be used to determine the μa(λ) and μs (λ) for calculating the chromophores and true color of tissues.

Measurement of the tooth color may be complex. For example, the reflectance mode spectroscopy may measure tooth whiteness. Colorimeters may provide color measurements in terms of three wavelengths of the visible spectrum. Spectrophotometers may measure color in one or more (e.g., all) wavelengths of the spectrum, which may render the wavelengths useful for color measurements (e.g., absolute color measurements) and/or color difference measurements. For example, CIE LAB color space is a color standard that may be used in dentistry. The perceived tooth color may be affected by optical properties of the dental structures, which may include translucency, surface texture, compositional structures' thickness, and/or illumination conditions.

Spectral transmission measurements can also be used for diagnosing hard tissue conditions by the device. early detection of various skin conditions. Transmission spectroscopy mapping of hard tissue can directly be used for detecting early caries or lesions. Polarized incident light around 1300 nm is known to transmit well through enamel. On the other hand, other wavelengths are often scattered into diffuse transmission. Cross-polarization prevents unscattered light from entering the detector and establishes a clear enamel boundary of the hard-tissue. However, if scattering centers exist in the enamel boundary, they will appear bright in an image. Other wavelengths are not as affected by caries/lesion scattering are already exit the other end of the hard tissue diffusely. By imaging a cross-polarized transmission ratio of the difference of the different wavelengths from the enamel and the lesion/caries regions, it is possible to map a 2D transillumination image of the lesion/caries region. This mapping can be sent to the user of the device or to a dental professional for understanding problem areas on the hard tissue.

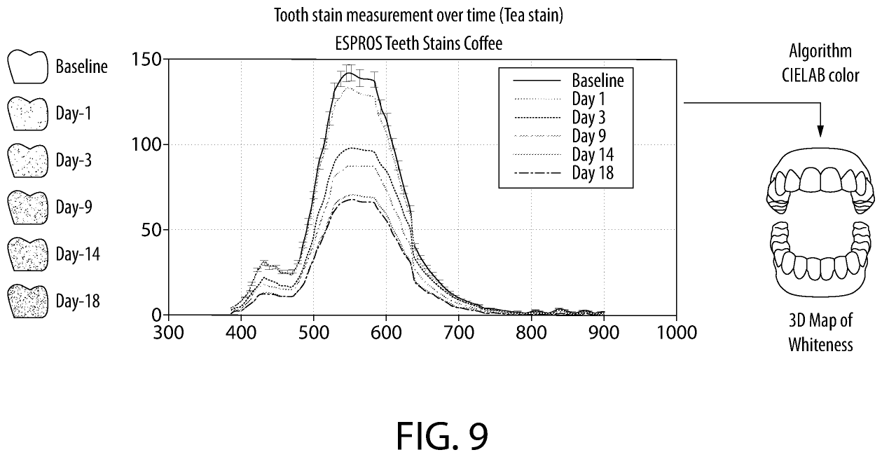

For measurement of the tooth color during device diagnostics, an algorithm based on visible spectral band of the matrix array spectral sensor may be used to provide the values of the values of L* (luminosity, or value), a* (quantity of red-green), b* (quantity of yellow-blue) color coordinates, or the L (luminosity), c (chroma), h (hue). FIG. 9 shows an example measurement of tooth color based on reflectance mode spectra and/or an algorithm that may convert the spectra to CIELAB. Spectral information collected by the matrix array multispectral sensor using the broadband white-light diode(s) may be used to calculate CIE LAB values (e.g., accurate CIE LAB values). The CIE LAB value may be calculated using one or more (e.g., three) color channels, although in examples CIE LAB value may be more accurate when using five or more color channels.

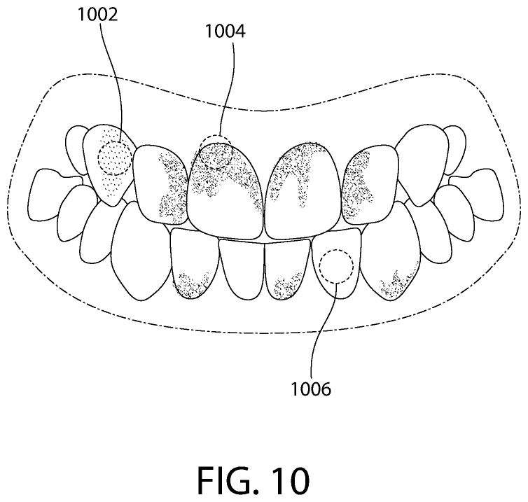

Quantitative light induced fluorescence (QLF) may be a sensitive non-contact method for the detection of enamel demineralization and/or dental caries. QLF may use the principle of mineral loss caused by the carious destruction of tooth enamel, which may measure a decrease in fluorescence intensity when exposed to blue light. QLF signal(s) may appear lower in intensity with respect to caries than sound enamel. The spectral sensor data may correspond to the QLF signal that may be utilized for estimating the enamel health. FIG. 10 shows example fluorescence images of the oral cavity using a 16-channel matrix array spectral sensor, where the tissue is excited by 405 nm radiation. The healthy enamel 1006 may be separated from carries 1002 and biofilm (such as plaque 1004), and the like. The healthy enamel may provide a baseline, for example, indicating tooth health. The index of enamel health and possible caries may be estimated, for example, based on the calculation of area under the curve of pixels determined to be from hard tissue. If the calculated index is below a predetermined health index threshold, the probability of caries may be high and/or may be reported to the user, sent to a third party, and the like.

Dental plaque may be detected using the 405 nm excitation wavelength and the emission of red fluorescence. The origin of red fluorescence in dental imaging may be from the porphyrins protein produced by bacterial metabolites in the anaerobic plaque. Red fluorescence may be used for dental plaque monitoring without any disclosing solutions. Red fluorescence spectra captured by the matrix array multispectral sensor (e.g., during diagnostics) may be used for measuring a plaque index. The index of dental plaque may be estimated based on the calculation of intensity per unit area of the red auto fluorescence spectral signal. To distinguish enamel fluorescence over dental plaque fluorescence, the fluorescence intensity ratios may be measured using the intensity centered at spectral channels, such as 510 nm and 620 nm, each with a known bandwidth (e.g., F510/F630). The ratio may be correlated with the clinical plaque score for discriminating different grades of caries and plaque from grade 0 (absence of plaque) to grades 1, 2, and 3 (high caries probability).

Bacteria on the tongue may cause an unpleasant smell (e.g., malodor). Monitoring red autofluorescence from the tongue may provide an indication of malodor and/or tongue health. In an example, a device (e.g., device 100) may excite the one or more portions of an oral cavity (e.g., tongue) using a UV/blue light source to induce porphyrin fluorescence. Red fluorescence may be collected from the tongue using the matrix array spectral sensor. The amount of porphyrin may be calculated by measuring the red fluorescence intensity per unit area. A score of 1 to 10 may be assigned for the porphyrin content on the tongue, which may be related to malodor. The device may track the average score over time, which may provide user feedback on changes on a tongue score.

Machine learning may be used (e.g., used solely or in combination with sensor output) to diagnose and/or score tissue health and oral hygiene. Machine learning (ML) algorithms may be supervised or un-supervised. For example, a deep-learning algorithm may find (e.g., automatically find) common features in data sets. The deep-learning algorithm may be trained (e.g., first be trained) on sets of images. In a training phase, the algorithm may be trained to identify common features of data in one or more (e.g., each) classified data sets. A set (e.g., large set) of sample images (such as more than 500 data sets) of conditions of interest may be acquired from the oral cavity. Sample images may be pre-acquired images from one or more (e.g., numerous) users.

The machine learning algorithm may be trained to determine whether the tissue is hard tissue or soft tissue. After the determination, the algorithm may access the white light or fluorescence excitation classification folder. The machine learning algorithm may classify the oral cavity (e.g., the entire oral cavity) on a pixel by pixel basis, which may give a final diagnostics. The machine learning algorithm may segment the mouth into one or more (e.g., different) zones and classify images (e.g., classify images separately). For white light images, the trained ML algorithm may calculate and/or map bleeding tissues, teeth whiteness, blood pressure, heart rat, blood flow, over brushing, ulcers, cracked teeth, inflammation, longitudinal gum recession, periodontitis, tonsillitis, melanin concentration, hydration, blood/tissue oxy-deoxygenation volume fraction, and the like. If fluorescence mode is detected, the ML algorithm may calculate and/or map plaque, potential caries/cavity spots, biofilm, and/or malodor from one or more portion of an oral cavity, such as a tongue.

In some examples of the ML algorithm, the white light and fluorescence data may be combined to improve health diagnostics. For example, hard tissue crack determination(s) may be more reliable if a hard tissue crack is detected in the white light images and confirmed in fluorescence images. Another example may be to detect potential carries in the fluorescence channel and detect hydration changes or discoloration in the white light/NIR channels of the matrix array multispectral sensor.

The device (e.g., device 100) may include a smart device application that may include a virtual assistant to improve oral health, device usage, and/or ease of understanding results. The virtual assistant may inform users of the meaning of results of diagnostics of blood concentration, tissue oximetry, caries detection, plaque detection, whiteness measurement, tooth enamel health measurements, and/or positional information of where problem areas may be located in the oral cavity. The assistant may provide brushing and/or product recommendations to improve overall health based on the individuals diagnostic. One or more (e.g., each) biometric measurement may be given a score (e.g., a standard index score) that may be related to health. For example, a standardized whiteness score may be achieved using CIE LAB, tissue and blood oxygen concentration may be mapped in percent oxygen, caries detection may be calculated on a scale of 0 to 4 (where 0 is healthy and 4 is likely positive for caries), and the like. Measured information may be relayed to the user through a display device and/or may be sent to a third party (e.g., an external server).

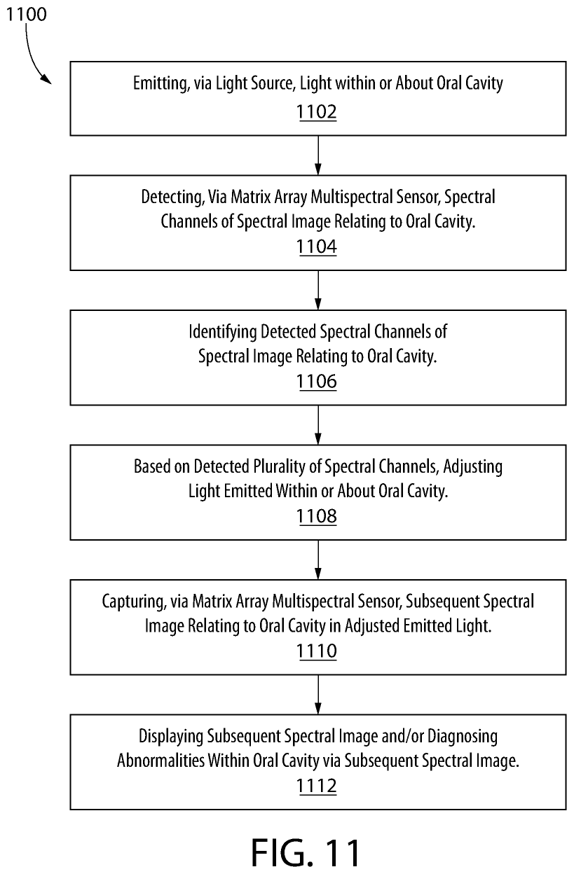

FIG. 11 describes an example process 1100 for using the device 100. At 1102, a light source may emit light within or about an oral cavity. The light source may emit light in a plurality of wavelengths. The light source may be a light emitting diode, superluminescent diode, laser, arc lamp, or the like. A multispectral sensor (e.g., matrix array multispectral sensor) may be optically coupled to the light source. At 1104, the matrix array multispectral sensor may detect a plurality of spectral channels of a spectral image relating to the oral cavity. Each of the plurality of spectral channels may allow transmission of a corresponding wavelength. At 1106, the detected plurality of spectral channels of the spectral image relating to the oral cavity may be received (e.g., identified).

A processor may perform one or more actions. For example, the detected plurality of spectral channels of the spectral image relating to the oral cavity may be received by a processor. The receiving of the spectral channels may include receiving a signal and/or data that may be indicative of the spectral channels. At 1108, based on the detected plurality of spectral channels (e.g., received detected plurality of spectral channels), the light source may adjust (e.g., caused to be adjusted by the processor) the light emitted within or about the oral cavity. The light source may adjust the light emitted to modify a signal to noise ratio and/or an image calibration. At 1110, the matrix array multispectral sensor may capture (e.g., the processor may cause the matrix array multispectral sensor to capture) a subsequent spectral image relating the oral cavity in the adjusted emitted light. At 1112, the subsequent spectral image may be displayed and/or the subsequent spectral image may be used to diagnose (e.g., diagnose via artificial intelligence techniques) abnormalities within the oral cavity, as described herein.

While the inventions have been described with respect to specific examples including presently preferred modes of carrying out the inventions, those skilled in the art will appreciate that there are numerous variations and permutations of the above described systems and techniques. It is to be understood that other embodiments may be utilized and structural and functional modifications may be made without departing from the scope of the present inventions. Thus, the spirit and scope of the inventions should be construed broadly as set forth in the appended claims.

1. An intraoral device for determining the oral health characteristics of an oral cavity, the intraoral device comprising:
a light source configured to emit light in a plurality of wavelengths within or about the oral cavity;
a matrix array multispectral sensor configured to detect a plurality of spectral channels of a spectral image relating to the oral cavity, each of the plurality of spectral channels allowing transmission of a corresponding wavelength; and
a processor configured to:
receive the detected plurality of spectral channels of the spectral image relating to the oral cavity;
based on the received detected plurality of spectral channels, cause the light source to adjust the light emitted within or about the oral cavity to modify at least one of a signal to noise ratio or an image calibration of a subsequent spectral image; and
cause the matrix array multispectral sensor to capture the subsequent spectral image relating to the oral cavity.

2. The intraoral device according to claim 1, further comprising one or more reflective elements positioned within or about the intraoral device, wherein the one or more reflective elements are configured to reflect portions of the oral cavity into a field of view of the matrix array multispectral sensor.

3. The intraoral device according to claim 2, wherein the one or more reflective elements comprise at least one of a flat reflector, a concave reflector, or a convex reflector, wherein the flat reflector is configured to prevent elongation or shrinkage of the spectral image relating to the oral cavity and the concave or convex reflectors are configured to adjust a magnification or a curvature of the spectral image relating to the oral cavity.

4. The intraoral device of according to claim 1, wherein the processor is further configured to:
determine a spectral transmission via the subsequent spectral image relating to the oral cavity in the adjusted emitted light; and
perform a determination of a hard tissue condition based on the determined spectral transmission.

5. The intraoral device of according to claim 1, wherein the matrix array multispectral sensor and the light source each comprise at least one polarizer configured to cross-polarize light emitted by the light source, the cross-polarizing of the light removing a specular reflection incident upon the matrix array multispectral sensor.

6. The intraoral device of according to claim 1, wherein the matrix array multispectral sensor is mounted on at least one of an outside wall of the intraoral device, a top of a mount of the intraoral device, or a bottom of the mount of the intraoral device.

7. The intraoral device of according to claim 1, further comprising one or more color calibration devices configured to perform a color calibration of the spectral image relating to the oral cavity.

8. The intraoral device according to claim 7, wherein the one or more calibration devices comprise a plurality of color calibration squares positioned in the field of view of the matrix array multispectral sensor.

9. The intraoral device of according to claim 1, wherein the matrix array multispectral sensor comprises a fisheye lens configured to provide an ultrawide and close-up large field of view image of the oral cavity.

10. The intraoral device of according to claim 1, wherein the intraoral device is a tray configured to be worn within the oral cavity.

11. The intraoral device of according to claim 1, wherein the processor is configured to cause the subsequent spectral image relating to the oral cavity in the adjusted emitted light to be displayed upon a display.

12. The intraoral device of according to claim 1, wherein the processor is configured to diagnose, via artificial intelligence techniques, a tissue relating to the oral cavity based on the subsequent spectral image relating to the oral cavity in the adjusted emitted light.

13. The intraoral device of according to claim 1, wherein the matrix array multispectral sensor comprises at least one of an RGB, RGB-IR, multispectral, or hyperspectral camera.

14. The intraoral device of according to claim 1, wherein the light source comprises one or more of a light emitting diode, superluminescent diode, laser, or arc lamp.

15. The intraoral device of according to claim 1, further comprising at least one of a bite bar, tooth rest, or tongue retractor configured to accept portions of the oral cavity.

16. The intraoral device of according to claim 1, wherein the light source comprises a broadband light source.

17. The intraoral device of according to claim 1, wherein the matrix array multispectral sensor is configured to detect fluorescence or a biofilm on or about a tooth within the oral cavity.

18. The intraoral device of according to claim 1, wherein the matrix array multispectral sensor is a distance sensor.

19. A method for determining the oral health characteristics of an oral cavity, the method comprising:
emitting, via a light source, light in a plurality of wavelengths within or about the oral cavity;
detecting, via a matrix array multispectral sensor, a plurality of spectral channels of a spectral image relating to the oral cavity, each of the plurality of spectral channels allowing transmission of a corresponding wavelength;
receiving the detected plurality of spectral channels of the spectral image relating to the oral cavity;
based on the received detected plurality of spectral channels, causing the light source to adjust the light emitted within or about the oral cavity to modify at least one of a signal to noise ratio or an image calibration of a subsequent spectral image; and
causing the matrix array multispectral sensor to capture a subsequent spectral image relating to the oral cavity.

20. The method of claim 19, wherein the steps of the method are preformed via an intraoral device worn within the oral cavity.
